(19) United States
(12) Patent Application Publication
(10) Pub. No.: US 2007/0031338 A1
(43) Pub. Date: Feb. 8, 2007

(54) Embolized cryoablation for treatment of tumors

(76) Inventor: Peter P. Zabinski, Melbourne, FL (US)

Correspondence Address: MELVIN K. SILVERMAN, 500 WEST CYPRESS CREEK ROAD, SUITE 500, FT. LAUDERDALE, FL, 33309, US

(21) Appl. No.: 11/496,265

(22) Filed: Jul. 31, 2006

Related U.S. Application Data
(60) Provisional application No. 60/704,938, filed on Aug. 2, 2005.

(51) Int. Cl. A61N 1/39; A61K 49/00; A61K 31/455

(52) U.S. Cl. 424/9.6; 514/355; 607/1

(57) ABSTRACT

An embolized-cryoablation method for treating a tumor of an organ is provided. The method includes inserting a catheter in a vascular pathway connected to a target region adjacent the tumor, and advancing the catheter through the vascular pathway to place a distal end portion of the catheter in the target region; injecting a liquid embolization material through the catheter into the target region; removing the catheter through the vascular pathway from the organ; inserting a cryoprobe laparoscopically into the target region, and placing a distal end portion of the cryoprobe within the target region; delivering a cryogen into and circulating the cryogen inside the cryoprobe for a period of time, thereby providing a cryotreatment to the target region of the organ. The combined embolization-cryoablation treatment reduces bleeding and enhances cell death, necrosis, or apoptosis in the tumor tissue.

### CROSS REFERENCE TO RELATED APPLICATION

This application claims the benefit under 35 USC 119 (e) of the provisional patent application Ser. No. 60/704,938, filed Aug. 2, 2005, which is herein incorporated by reference in its entirety.

### FIELD OF THE INVENTION

The present invention relates to a method for treatment of tumors, and, in particular, a method for treating tumors in kidney or other organs using embolized-cryotreatment.

### BACKGROUND OF THE INVENTION

Cryoablation is a method of in situ tumor ablation in which subfreezing temperatures are delivered through penetrating or surface cryoprobes in which a cryogen is circulated. Thermally conductive material allows cooling at the probe tip while the shaft and delivery hoses are insulated. Irreversible tissue destruction occurs at temperatures below −20° C. to −30° C. Cell death is caused by direct freezing, denaturation of cellular proteins, cell membrane rupture, cell dehydration, and ischemic hypoxia. Cryoablation as large as 6-8 cm in diameter can be provided safely. Cryoablation is the oldest of the local thermal ablation techniques. Cooper first suggested its use for treating liver tumors in 1963, since then, there have been multiple clinical reports detailing its use for the treatment of primary and secondary malignant hepatic tumors and kidney tumors.

Cryosurgery for the treatment of kidney cancers is gaining popularity nationwide. Urologists are seeing a growing number of small renal cancers caught early, before symptoms, because of better imaging and more proactive consumer driven body scans. 15-25% of kidney cancer patients have metastatic disease at any time of diagnosis. For patients with only one kidney or deceased renal function, where removal of a portion or the entire kidney would mean dialysis for life, cryosurgery holds great promise for these patients.

Cryoablation of kidney tumor is a laparoscopic procedure during which the urologist, guided by ultrasound, inserts small probes into the kidney tumor. Laparoscopic ultrasound is a new surgical imaging method which provides direct contact imaging of organs with high frequency ultrasound. In the procedure, the probe tips are cooled to freezing temperatures, destroying tumor cells. An advantage of this minimally invasive technique is that the urologist can watch the freezing process in real time through ultrasound monitoring, making it easier to focus treatment just on the tumor cells. The procedure is used on small tumors less than 4 cm in diameter and it is approved by the U.S. Food and Drug Administration.

Embolization is defined as the therapeutic introduction of various substances into the circulation to occlude vessels, either to arrest or prevent hemorrhaging, to devitalize a structure, tumor, or organ by occluding its blood supply, or to reduce blood flow to an arteriovenous malformation.

Embolization can be used for achieving three different therapeutic goals: (1) an adjunctive goal, for example, preoperative, adjunct to chemotherapy or radiation therapy; (2) a curative goal, for example, definitive treatment such as that performed in cases of aneurysms, arteriovenous fistulae (AVFs), arteriovenous malformation (AVMs), and traumatic bleeding; and (3) a palliative goal, for example, relieving symptoms, such as of a large AVM, which cannot be cured by using embolotherapy alone.

Medical conditions treated by using embolotherapy can be grouped as follows: (1) vascular anomalies, for example, AVM, AVF, venous malformation (VM), lymphatic malformation (LM), and hemangioma; (2) hemorrhage, for example, pseudoaneurysms and gastrointestinal tract, pelvic, posttraumatic, epistaxis, and hemoptysis bleeding; and (3) other conditions, for example, tumors, varicoceles, and organ ablation.

Indications for embolotherapy in neoplastic conditions include preoperative embolization and palliative embolization, which alleviates symptoms, reduces further dissemination, and increases the response to other treatment modalities (for example, radiation therapy). Embolotherapy can be used for many types of malignant tumors. Renal malignancy is the most common type of tumor treated with embolotherapy. In particular, tumors extending into the hilum or other adjacent structures for which surgical removal is difficult are treated by using embolotherapy. In these patients, prior embolization of the tumor shrinks the mass and minimizes blood loss during surgical removal.

Unresectable tumors can be made operable by means of embolotherapy. If the entity is in its end stage (disseminated metastatic deposits), the technique can be used for palliation to control pain and hematuria. Other reported malignancies in which embolotherapy has been used include pelvic malignancies and bone tumors. Hemorrhage resulting from malignancy or radiotherapy (for example, due to radiation cystitis) can be controlled by using embolotherapy.

Materials that have been used in embolization include coils, ethanol, sodium tetradecyl sulfate, cyanoacrylate, polyvinyl alcohol (PVA), microspheres, and gelatin sponge (Gelfoam), among others. Other less commonly or previously used materials include balloons, microfibrillar collagen (Avitene), ethiodized oil (Ethiodol), autologous materials, ethylene vinyl alcohol, alginates, phosphoryl choline, sodium morrhuate, hot contrast material, and 50% dextrose.

Despite recent development of the cryoablation technique for the treatment of primary or secondary malignant renal and hepatic tumors, further development of cryoablation technology still remains as a strong clinical need to improve treatment efficiency and to improve cancer patients' survival rate. Although both cryoablation and embolization have been separately used for treating tumors, embolization has not been used together with cryoablation for treatment of tumors.

### SUMMARY OF THE INVENTION

In one embodiment, the present invention is directed to an embolized-cryoablation method for treating a tumor of an organ. The method comprises the steps of inserting a catheter in a vascular pathway connected to a target region adjacent the tumor of the organ, and advancing the catheter through the vascular pathway to place a distal end portion of the catheter in the target region; injecting a predetermined amount of a liquid embolization material through the catheter into the target region; removing the catheter through the vascular pathway from the organ; inserting a cryoprobe laparoscopically into the target region, and placing a distal end portion of the cryoprobe within the target region; and delivering a cryogen into and circulating the cryogen inside the cryoprobe for a first period of time, thereby providing a cryotreatment to the target region of the organ.

Preferably, the method further comprises delivering the cryogen into and circulating the cryogen inside the cryoprobe for a second period time, thereby providing a second cryotreatment to the target region.

Moreover, the method further comprises inducing a rapid thaw at the target region after each cryotreatment, to further enhance the damages to the tumor tissue.

In a further embodiment, the method of the present invention method can further comprise infusing a chemotherapeutic agent into the target region together with the liquid embolization material. The chemotherapeutic agent can be selected to be specific to a particular type of tumor.

The embolized-cryoablation method of the present invention is particularly suitable for treating kidney tumors, and can also be used for treating tumors in liver, brain, breast, or prostate.

The advantages of the present invention will become apparent from the following descriptions with the accompanying examples.

### DETAILED DESCRIPTION OF THE INVENTION

In one embodiment, the method of the present invention provides a method of treating a tumor of a patient. More specifically, the method, or treatment process, comprises the following steps:

(a) inserting a catheter in a vascular pathway connected to a target region adjacent the tumor of the organ, and advancing the catheter through the vascular pathway to place a distal end portion of the catheter in the target region;

(b) injecting a predetermined amount of a liquid embolization material through the catheter into the target region;

(c) removing the catheter through the vascular pathway from the organ;

(d) subsequently, inserting a cryoprobe laparoscopically into the target region, and placing the distal end portion of the cryoprobe within the target region;

(e) delivering a cryogen into the cryoprobe and circulating the cryogen inside the cryoprobe, thereby providing a cryotreatment of the target region of the organ at a predetermined temperature for a period of time; and

(f) removing the cryoprobe from the organ.

The treatment process can further include a step of thawing the target region, and then subsequently providing a second cryotreatment of the target region for a second period time, as described in more detail hereinafter, prior to removing the cryoprobe.

Two types of thawing process can be used for the purpose of the present invention. One process is a passive thaw, which is a spontaneous thawing process of the ice ball formed in the cryotreated target region. Another process is a rapid thaw. Rapid thaw is achieved by heating the ice ball formed using an embedded heating element inside the tip of a cryoprobe. Rapid thaw typically takes from about 5 minutes to about 15 minutes. The time of thaw depends on the size of the tumor being treated, or the size of the ice ball formed. It has been known that the rapid thaw can cause apoptosis and cell lysis, which can further enhance damages to the tumor tissue. In a preferred embodiment, a rapid thaw is used after the first and the second cryotreatments of the method of the present invention.

The method of the present invention is suitable for treatment of renal tumors, particularly for renal tumors having a size about 4 cm or less, and is described hereinafter with examples of treating such conditions. However, it should be understood that the method can also be used for treatment of tumors in other organs in human or animal body, such as liver, brain, breast, and prostate.

As used herein, the term “target region” refers to a defined volume or mass of tissue in an organ of the human or animal body, which includes therein the tumor to be treated. As used herein, the term “cryotreatment” refers to a treatment of body tissue with a very low temperature generated by a cryogen, which includes cryoablation. Cryoablation refers to the application of a very low temperature to body tissue to such a degree so as to cause cell death, necrosis, or apoptosis in the tissue. As used herein, the term “embolized-cryotreatment” refers to the procedure that performs an embolization first on a target region and subsequently performs a cryotreatment to the same target region, which includes “embolized-cryoablation”.

The liquid embolization material used herein includes, but is not limited to, ethiodized oil, microfibrillar collagen, autologous materials, ethylene vinyl alcohol, alginates, phosphoryl choline, sodium morrhuate, contrast material, and dextrose. Preferably, ethiodized oil is used. Ethiodized oil functions as both an embolization material and a radio-opaque diagnostic agent.

Furthermore, an image enhancement agent can further be included in the liquid embolization material. For example, a combination of ethiodized oil with methylene blue can be used to enhance the ultrasound image for effective monitoring of the treatment process. In a preferred embodiment, typically about 1.5 ml to about 3 ml of ethiodized oil in the form of Ethiodol is used for the embolization step. Optionally, about 1 ml to about 2 ml of 1% methylene blue aqueous solution can be used together with the Ethiodol for enhancing the ultrasound image for the purpose of monitoring the treatment process.

Ethiodol is a sterile injectable radio-opaque diagnostic agent commonly used for hysterosalpingography and lymphography, which is commercially available from Savage Laboratories, Melville, N.Y. It contains 37% iodine (475 mg/ml) organically combined with ethyl esters of the fatty acids (primarily as ethyl monoiodostearate and ethyl diiodostearate) of poppyseed oil, and stabilized with poppyseed oil, 1%. The precise structure of Ethiodol is unknown at this time. Ethiodol is a straw to amber colored, oily fluid, which possesses a greatly reduced viscosity (1.280 specific gravity at 15° C. yields viscosity of 0.5-1.0 poise). This high fluidity provides flexibility for radiographic use. 1% methylene blue aqueous solution is commercially available as a sterile solution for slow intravenous administration, such as Methylene Blue Injection, USP, 1%, manufactured by Faulding Pharmaceutical Co., Paramus, N.J.

The cryogen used can be any number of fluids suitable for stable compression to pressures on the order of 10 psig to up to 6000 psig. Preferential examples of such fluids are nitrous oxide (N₂O), nitrogen (N₂), argon, or AZ-20. Several cryosurgical systems are commercially available. Cryotech/Candela (Wayland, Mass.) uses liquid nitrogen under pressure. Cryomedical Sciences (Rockville, Md.) utilizes super-cooled liquid nitrogen, and EndoCare (Irvine, Calif.) uses argon gas as the cryogenic material. Multiple probes in varying sizes and configurations are provided with the instrument systems by the manufacturers. In a preferred embodiment the present invention, argon is used for cooling, and helium is used for heating during the rapid thaw.

The catheter for delivering liquid embolization materials and the cryoprobe can be flexible or rigid. The catheter can be constructed of a variety of materials, including plastics and both ferrous and non-ferrous metals, and preferably have diameters of 2 to 7 French. It should be understood that various commercial available cryoprobes that have different configurations and sizes can be used for the purpose of the present invention. In general, a cryoprobe is a catheter that has an outer body having a proximal end portion and a distal end portion, and an inner body within the outer body. The distal end portion typically encloses a cooling chamber which is in fluid communication with the inner body. When it is used, the proximal end portion is connected to the source of the cryogen, and the distal end portion is first inserted into the tumor. When the cryogen is introduced into the cooling chamber through the inner body, it circulates inside the cooling chamber to freeze the tissue around the distal end portion of the cryoprobe. During this process, an ice ball is formed in the surrounding tissue. The cryoprobe can be heated to provide a rapid thaw between the first and the second cryotreatments, and prior to the removal of the cryoprobe.

Cryotreatment, or cryoablation, in the method of the present invention is a laparoscopic procedure. Ultrasound is used for guiding the procedure. Depending on the tumor size, one to four probes can be placed within the tumor with the distal end portions of the cryoprobes penetrating about 5 to 10 mm beyond the tumor margin. The cryogen (at about −160° C. when argon is used) is circulated within the cryoprobe. The ice ball is visualized as an echogenic, expanding, hemispherical rim. Freezing is continued until the ice ball extends through the tumor and into the adjacent normal tissue, typically with the goal of achieving an ablation margin about 5 to 10 mm. It should be understood that varying shapes of ice ball can be formed, having linear, cylindrical, ellipsoidal, toroidal, or curved topologies. Typically, the temperature of the target region under treatment is in a range from about −20° C. to about −60° C., preferably, below −40° C. This first freeze typically takes about 4 to about 15 minutes and is followed by a rapid thaw, or a spontaneous thaw. Then, a second freeze is obtained by circulating the cryogen again within the probes. The second freeze reaches the same temperature of the treated target region for a time period similar to, or shorter than, the first freeze. Preferably, the second freeze reaches and slightly exceeds the original cryoablation margin. After the second freeze, the cryoprobes are heated again and removed from the organ. After the removal of the cryoprobe(s), the puncture wound is observed for hemostasis. If active bleeding is observed, the wound is packed by suitable materials to achieve hemostatis. Commonly used materials for packing the wound include gel foam, oxidized regenerated cellulose hemostatic agent, such as Surgicel® from J & J Health Care Systems Inc., Piscataway, N.J., and fibrin sealant, such as TISSEEL VH fibrin sealant from Baxter Healthcare Corporation, Glendale, Calif.

The method described above can be a continuous process, which provides embolization of the target region inclusive of the tumor, and then performs cryoablation of the target region subsequently. However, the embolization and the cryoablation can be two sequential steps with a period of time in-between. Clinically, it is common that a patient can have a separate embolization procedure performed first, and during the procedure the doctor monitors and investigates the tumor size and conditions. If the tumor condition is suitable for cryoablation, then the cryoablation procedure can be performed subsequently. Preferably, the period of time between the two procedures is in a range from about 1 to about 8 hours, therefore, the patient can be treated in the hospital in one day. However, it has been found that the cryoablation step can be performed from about 24 hours to about 72 hours, subsequent to the embolization of the target region, preferably within 24 hours.

The embolized-cryoablation method of the present invention has several advantages over the existing cryoablation method. It has been found that performing a cryoablation to a target region subsequent to the embolization of the same region substantially enhances cell death, necrosis, or apoptosis in the tumor tissue. This method first occludes vascular system inside and surrounding the tumor, then ablates the tumor tissue by cryotreatment. After this procedure, not only the tumor tissue is damaged by the cryotreatment, but also no blood supply is available to the region to support the recovery.

Furthermore, it has been found that the embolized-cryoablation method of the present invention substantially reduces bleeding commonly caused by freezing occurred in the cryoablation procedure. As described above, the first embolization step of the instant method occludes the vascular system inside and surrounding the tumor, therefore, bleeding through the vascular system in the target region is inhibited by embolization material penetrated into the vascular system. Some patients who have a high risk of severe bleeding because of the tumor size and location thereof and are not suitable for the traditional cryoablation, can be treated using the method of the present invention. This technical advantage broadens the scope of suitable candidates for the minimally invasive cryotreatment, and ultimately benefits the cancer patients who are in need for an effective surgical treatment.

Moreover, the embolized-cryoablation method of the present invention can be used for the renal tumor patients who have marginal kidney function. As described above, the method uses ethiodized oil as the radio-opaque agent for monitoring the procedure by ultrasound, optionally in combination with methylene blue. This allows an effective treatment of the renal tumor, without substantial degradation of the patient's kidney function.

Examples 1 to 3 provide clinical examples of the method of the present invention used in treating three renal cancer patients. Clinical follow up of 18 kidney cancer patients who have been treated using the method of the present invention for up to 18 months has showed no reoccurrence of the cancer. This clinical result is significant in comparison to an average reoccurrence rate of about 5% to 10% in the kidney cancer patients treated by traditional cryoablation, or by partial nephrectomy.

In a further embodiment, the embolized-cryotreatment of the present invention can be used in conjunction with the local use of chemotherapeutic agents. As used herein, “chemotherapeutic agents” refers to certain drugs, chemicals, or substances, such as apoptosis enhancers, used to further enhance the effects of embolized-cryotreatment. In this embodiment, the chemotherapeutic agent can be introduced to the target region through the vascular system in a mixture with the liquid embolization material.

It is known clinically that systemic chemotherapy is generally not helpful to renal cancers, because the concentration of the chemotherapeutic agent that reaches the renal tumor is inadequate for therapeutic purpose. The direct local introduction of the chemotherapeutic agent can increase the drug concentration in the tumors by 10 to 100-fold in comparison with the drug concentration achieved through systemic infusion. Furthermore, in the mixture with the embolization material, in other words using the embolization material as a carrier of the chemotherapeutic agent, the dwell time of the drug is prolonged from hours to weeks. Therefore, a further combination of chemotherapeutic agents with the embolized-cryotreatment described above can further enhance the overall effect against the renal tumor. The same is also applicable to the treatment of tumors of other organs.

In an exemplary embodiment, sorafenib or under the tradename of Nexavar®, can be used in the embolization step of the instant method. Sorafenib is a chemotherapy agent specific to kidney cancer. The chemical name of this medicine is 4-[4-[[4-chloro-3-(trifluoromethyl)phenyl]carbamoylamino]phenoxy]-N-methyl-pyridine-2-carboxamide, with a formula of C₂₁H₁₆CIF₃N₄O₃.

The following examples are illustrative of the invention and are in no way to be interpreted as limiting the scope of the invention, as defined in the claims. It will be understood that various other ingredients and proportions may be employed, in accordance with the proceeding disclosure.

### EXAMPLE 1

A male patient was diagnosed with a 2 cm hypo-vascular renal cell carcinoma involving the lateral upper pole of the left kidney. A super selective renal artery embolization and a subsequent laparoscopic cryoablation were performed on the patient, according to the method of the present invention.

The patient was given intravenous conscious sedation with Versed (Hoffmann-La Roche Inc., Nutley, N.J.) and fentanyl citrate (AstraZeneca Pty Ltd., North Ryde, NSW, Australia). The right common femoral artery was accessed retrograde with a 4-French micropuncture catheter. This was then exchanged for a 5-French arterial sheath. A selective and super selective left renal arteriogram was performed first. The finding of the arteriogram indicated that there was splaying of the arcuate arteries within the lateral superior upper pole of the left kidney. There was no angiographic evidence of hyper-vascularity within the tumor nodule or abnormal parenchymal blush, and no arterial venous shunting.

Then a superselective embolization of left subsegmental renal artery was performed, with fluoroscopic guidance. A microcatheter of 3 French was positioned within a fourth order subsegmental/arcuate branch of the left renal artery supplying the left upper lateral renal cell carcinoma nodule. Under intermittent fluoroscopic observation 1.5 ml of Ethiodol (Savage Laboratories, a Division of Altana Inc. Melville, N.Y.) was instilled via the microcatheter within the vascular distribution supplying the tumor. There was a significant uptake of the Ethiodol by the renal cell carcinoma. Then the microcatheter was removed and hemostasis was obtained.

As this patient's condition was suitable for the embolized-cryoablation treatment, the patient was transferred to an operation room in the same hospital. After satisfactory general anesthetic was obtained, a cryoablation was performed on the patient. The instrument used is a Endocare Cryoablation System, manufactured by Endocare Inc., Irvine, Calif. Utilizing laparoscopic ultrasound the tumor was then identified, since it had been embolized with 1.5 ml of Ethiodol. Using the standard laparoscopic technique, the Gerota fascia was entered. Two 3-mm trial probes were placed and were extended 1 cm beyond the tumor margin, which were well visualized under ultrasound guidance. A first 5-minute freeze was obtained, and followed by a rapid thaw of about 5 minutes. Then a second 4-minute freeze was obtained, and followed by another rapid thaw of about 5 minutes. Under the ultrasound guidance, the ice ball extended well beyond the tumor rim, which was easily identified with the Ethiodol. The puncture wounds were visualized approximately 10 minutes after the probes were removed, and no active bleeding was observed. The 10-mm port was then closed utilizing the Carter-Thomason needle closure device. Skin staples were applied, and the patient was taken to the recovery room in satisfactory condition.

### EXAMPLE 2

A 66 year old female patient was diagnosed with a 2.0 cm×2.5 cm exophytic lesion within the left kidney by abdomen CAT scan, with no adenopathy or liver metastasis. The patient had a past medical history of hypertension and chronic renal insufficiency. A selective renal artery embolization and a subsequent laparoscopic cryoablation were performed on the patient, according to the method of the present invention.

The patient first underwent a left renal selective angiography and selective embolization of the renal mass with Ethiodol. The next day, the patient was taken to the operation room for left renal biopsy, which indicated possible renal cell carcinoma, and a cryoablation was immediately performed using the Endocare Cryoablation System.

After satisfactory general anesthetic was obtained, the patient was placed in a dorsal lithotomy position and prepared in the usual manner. The pneumoperitoneum was then established under the left subcostal margin in the anterior clavicular line, and utilizing a Visiport™ Optical Trocar the abdominal cavity was entered. The splenic flexure was carefully mobilized and reflected medially, and perirenal fat was carefully dissected from the tumor. Then utilizing 5-mm lens and the laparoscopic ultrasound two 5 mm probes were inserted approximately 1.5 cm to 2 cm in distance from each other directly into the tumor, extending 4 cm in depth from the margin of the kidney, which was 1 cm beyond the margin of the tumor. A first 5-minute freeze was given, and followed by a rapid thaw of about 5 minutes. Then a second 5-minute freeze was given, and followed by another rapid thaw of about 5 minutes. 3 to 4 minutes after the second thaw, the cryoprobes were removed. A small amount of bleeding was observed at the port, which was packed with Surgicel and no further bleeding occurred in about 15 minutes. The 12 mm port was closed by sutures.

An abdomen CAT scan was performed on the patient 13 month after the embolized-cryoablation treatment. Evaluation of the kidneys revealed a cortical scar at the lateral lower pole cortex of left kidney consistent with the patient's cryoablation therapy history, with no evidence of residual disease.

### EXAMPLE 3

A 77 year old male patient was diagnosed with a 3.7 cm×3.3 cm of renal mass at apex of the right kidney by CAT scan.

The patient first underwent a right renal selective angiography and selective embolization of the renal mass with Ethiodol as described above. The patient was taken to the operation room in the same day for right renal biopsy. After general anesthetic was obtained, the patient was placed in the modified right flank position at approximately 45° and prepared in the usual manner. The pneumoperitoneum was then initiated in the right subcostal margin and the peritoneal cavity was then entered with a 12-mm port utilizing a Visiport™ Optical Trocar. The right colon was mobilized including hepatic flexure, and the kidney was mobilized from the entire lateral aspect superiorly and medially. The Gerota fascia was then entered, and the superomedial aspect of the kidney and the perirenal fat was then carefully dissected and removed. The renal mass was identified; it was biopsied with a Tru-Cut needle, and the frozen section revealed a renal cell carcinoma. A cryoablation was performed immediately using the Endocare Cryoablation System. The edge of the liver was reflected medially utilizing a Jarit retractor. The cryoprobe was then placed within the tumor, extending 4 cm in depth from the margin of the kidney, which was 1 cm beyond the margin of the tumor. A first 10-minute freeze was given, and followed by a rapid thaw of about 5 minutes. Then a second 10-minute freeze was given, and followed by another rapid thaw of about 5 minutes. Then the cryoprobes were removed. A small amount of bleeding was observed at the port. Tisseel was applied along with Surgicel, and the bleeding abated. Observation was made that revealed no damage to liver or abdomen. The port was closed by sutures.

An abdomen CAT scan was performed on the patient 3 month after the embolized-cryoablation treatment. Evaluation of the kidneys revealed a benign appearance cystic mass at the upper pole right kidney, a sequela of prior embolization and cryoablation. The CAT scan revealed no evidence of residual disease.

While there has been shown and described the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention as set forth in the Claims appended herewith.

What is claimed is:

1. A method of treating a tumor of an organ, comprising the steps of:
(a) inserting a catheter in a vascular pathway connected to a target region adjacent said tumor of said organ, and advancing said catheter through said vascular pathway to place a distal end portion of said catheter in said target region;
(b) injecting a predetermined amount of a liquid embolization material through said catheter into said target region;
(c) removing said catheter through said vascular pathway from said organ;
(d) inserting a cryoprobe laparoscopically into said target region, and placing a distal end portion of said cryoprobe within said target region; and
(e) delivering a cryogen into and circulating said cryogen inside said cryoprobe for a first period of time, thereby providing a cryotreatment to said target region of said organ.

2. The method of claim 1 further comprising thawing said target region after step (e), prior to removing said cryoprobe.

3. The method of claim 2 further comprising delivering said cryogen into and circulating said cryogen inside said cryoprobe for a second period time, thereby providing a second cryotreatment to said target region.

4. The method of claim 3 further comprising thawing said target region after said second cryotreatment, prior to removing said cryoprobe.

5. The method of claim 4, wherein said thawing is a rapid thaw induced by heating inside said distal end portion of said cryoprobe.

6. The method of claim 1, wherein said target region under said cryotreatment has a temperature below −20° C.

7. The method of claim 1, wherein said cryotreatment cryoablation.

8. The method of claim 1, wherein said liquid embolization material further comprises an image enhancement agent.

9. The method of claim 8, wherein said image enhancement agent is methylene blue.

10. The method of claim 1 further comprising infusing a chemotherapeutic agent into said target region together with said liquid embolization material.

11. The method of claim 10, wherein said chemotherapeutic agent is 4-[4-[[4-chloro-3-(trifluoromethyl)phenyl]carbamoylamino]phenoxy]-N-methyl-pyridine-2-carboxamide (sorafenib).

12. The method of claim 1, wherein said organ is kidney.

13. The method of claim 1, wherein said organ is liver, brain, breast, or prostate.

14. A method of treating a tumor of an organ, comprising the steps of:
(a) inserting a catheter in a vascular pathway connected to a target region adjacent said tumor of said organ, and advancing said catheter through said vascular pathway to place a distal end portion of said catheter in said target region;
(b) injecting a predetermined amount of a liquid embolization material through said catheter into said target region;
(c) removing said catheter through said vascular pathway from said organ;
(d) inserting a cryoprobe laparoscopically into said target region, and placing a distal end portion of said cryoprobe within said target region;
(e) delivering a cryogen into and circulating said cryogen inside said cryoprobe for a first period of time, thereby providing a cryotreatment to said target region of said organ;
(f) thawing said target region;
(g) delivering said cryogen into and circulating said cryogen inside said cryoprobe for a second period time, thereby providing a second cryotreatment to said target region; and
(h) thawing said target region again.

15. The method of claim 14, wherein said thawing is a rapid thaw induced by heating inside said distal end portion of said cryoprobe.

16. The method of claim 14, wherein said liquid embolization material further comprises an image enhancement agent.

17. The method of claim 15, wherein said image enhancement agent is methylene blue.

18. The method of claim 14 further comprising infusing a chemotherapeutic agent into said target region together with said liquid embolization material.

19. The method of claim 1, wherein said organ is kidney.

20. The method of claim 1, wherein said organ is liver, brain, breast, or prostate.
